(19) United States
(12) Patent Application Publication
(10) Pub. No.: US 2021/0393719 A1
(43) Pub. Date: Dec. 23, 2021

(54) POWDERED BOTANICAL EXTRACT PREPARATIONS AND FORMULATIONS THEREOF

(71) Applicant: Sonny Fong, Pointe-Claire, Quebec (CA)

(72) Inventors: Sonny Fong, Pointe-Claire, Quebec (CA); Diego Castillo-Guimond, Laval, Quebec (CA)

(21) Appl. No.: 17/283,921

(22) PCT Filed: Oct. 18, 2019
(86) PCT No.: PCT/CA2019/051477

§ 371 Date: Apr. 8, 2021

Related U.S. Application Data
(60) Provisional application No. 62/748,266, filed on Oct. 19, 2018.

(51) Int. Cl. A61K 36/185; A61K 9/16; A61K 9/00

(52) U.S. Cl. CPC A61K 36/185; A61K 9/1694; A61K 2236/15; A61K 9/0014; A61K 9/1617

(57) ABSTRACT

The present invention provides a powdered botanical extract preparation comprising an evenly distributed dispersion of a botanical extract in a methylsulfonylmethane matrix, processes for preparing the preparations, and dosage forms comprising the preparations.

### FIELD OF THE INVENTION

The present invention pertains to the field of botanical extracts, and in particular to formulations comprising a powdered botanical extract preparation.

### BACKGROUND

Botanical materials have long been known to be a source of medicinal substances, from traditional medicines used, for example, in traditional Chinese or Ayurvedic medicinal practice, to compounds that can be encountered on a daily basis, for example, caffeine, menthol and nicotine, to compounds that have been discovered through modern drug discovery processes, such as the anti-cancer medicines taxol and vinblastine.

The botanical materials may be used directly as herbal medicines. Alternatively, the botanical materials may undergo a treatment process to provide access to the medicinal substance. Such processes can include any combination of extraction, distillation, fractionation, concentration, fermentation, or other chemical or biological methods, and the resulting medicinal products can be in the form of extracts, decoctions, tinctures, essential oils and others.

Cannabinoids are compounds derived from Cannabis plants, including, for example, Cannabis sativa, an annual plant in the Cannabaceae family. The plant contains about 60 cannabinoids, including tetrahydrocannabinol (THC) and cannabidiol (CBD), which can be used for the treatment of a wide range of medical conditions, including glaucoma, AIDS wasting, neuropathic pain, treatment of spasticity associated with multiple sclerosis, fibromyalgia and chemotherapy-induced nausea. Additionally, these compounds have been reported to exhibit a therapeutic effect in the treatment of allergies, autoimmune disorders and inflammation, infection, epilepsy, depression, migraine, bipolar disorders, anxiety disorder, drug dependency and withdrawal syndromes, anorexia, cachexia, and dermis regulation. THC is particularly effective as an anti-emetic drug and is administered to curb emesis, a common side effect accompanying the use of opioid analgesics and anesthetics, highly active anti-retroviral therapy and cancer chemotherapy.

Cannabinoids are lipophilic and potentially acid-labile compounds. Because of their hydrophobic nature, cannabinoids are poorly absorbed systemically from oral dosage forms because of the poor dissolution of cannabinoids in the aqueous environment of gastrointestinal tract. Oral formulations of cannabinoids, therefore, exhibit low bioavailability.

An optimal oral dosage form of cannabinoids is not yet available due to the substantial ‘first pass’ metabolic effect which limits the oral bioavailability of cannabinoids to 6% [Karschner et al., Plasma Cannabinoid Pharmacokinetics Following Controlled Oral Δ9-Tetrahydrocannabinol and Oromucosal Cannabis Extract Administration. Clin Chem 2011 January; 57(1): 66-75]. Currently, medical Cannabis is administered primarily through smoking or, alternatively, consumed orally in the form of oil, cookies, chocolates, etc. The majority of these products are not standardized, i.e. cannabinoids compositions and doses are uncontrolled, and demonstrate major disadvantages of poor bioavailability resulting in administration of very high cannabinoid doses and increased side effects, high dosing frequency, high variability, low patient compliance, and short product self-life due to formulation instability.

Although Cannabis extracts are a convenient source of cannabinoids, due to the oily, tar-like nature of the extracts, they are difficult to formulate into standardized dosage forms without the use of multistep processes typically employing large volumes of organic solvents and specialized equipment.

Therefore there is a need for processes for preparing botanical extract preparations, and in particular Cannabis extract preparations, that are easy to handle and formulate into convenient and standardized dosage forms, and that does not require large volumes of organic solvents or specialized equipment. There is also a need for a Cannabis extract preparation that has the additional advantage of increased cannabinoid bioavailability.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide powdered botanical extract preparations and formulations thereof. In accordance with an aspect of the present invention, there is provided a process for preparing a powdered botanical extract preparation, the process comprising the steps of: a) providing a solid mixture of botanical extract/MSM; and b) milling the solid botanical extract/MSM mixture to provide the powdered botanical extract preparation, wherein the powdered botanical extract preparation comprises an evenly distributed dispersion of the botanical extract in a MSM matrix.

In one embodiment, the solid botanical extract/MSM mixture is formed by the following steps: i) providing a molten methylsulfonylmethane (MSM) phase, ii) adding a botanical extract to the molten MSM phase to form a liquid mixture of botanical extract/MSM, iii) stirring the liquid botanical extract/MSM mixture until the botanical extract has dissolved in the MSM to form a single phase botanical extract/MSM solution, and iv) cooling the single phase botanical extract/MSM solution to form the solid botanical extract/MSM mixture.

In another embodiment, the solid botanical extract/MSM mixture is formed by the following steps: i) providing a solution of a botanical extract in a solvent; ii) adding methylsulfonylmethane (MSM) to the botanical extract solution to form a botanical extract/MSM/solvent mixture, iii) stirring the botanical extract/MSM/solvent mixture until a single phase botanical extract/MSM solution is formed, d) removing the solvent from the homogeneous botanical extract/MSM/solvent phase to form the solid botanical extract/MSM mixture

In a preferred embodiment, the botanical extract is a Cannabis extract.

In accordance with another aspect of the present invention, there is provided a powdered botanical extract preparation prepared using processes in accordance with the present invention

In accordance with another aspect of the present invention, there is provided a powdered Cannabis extract preparation comprising an evenly distributed dispersion of a Cannabis extract in a methylsulfonylmethane matrix.

In accordance with another aspect of the present invention, there is provided an oral dosage form comprising a preparation in accordance with the present invention, and one or more pharmaceutically acceptable excipients or carriers.

In accordance with another aspect of the present invention, there is provided a topical formulation comprising a preparation in accordance with the present invention, and one or more pharmaceutically acceptable excipients or carriers.

### BRIEF DESCRIPTION OF THE FIGURES

FIG. 1 is a photograph of a Cannabis extract prior to undergoing a process in accordance with the present invention.

FIG. 2 is a photograph of a mixture of a Cannabis extract and MSM prior to solidification in a process in accordance with the present invention.

FIG. 3 is a photograph of the powdered Cannabis extract preparation after undergoing a process in accordance with the present invention.

FIG. 4 is a schematic depiction of a process in accordance with one embodiment of the present invention.

FIG. 5 is a schematic depiction of a process in accordance with one embodiment of the present invention.

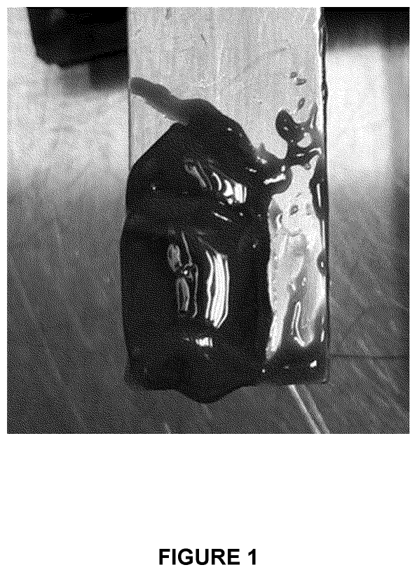

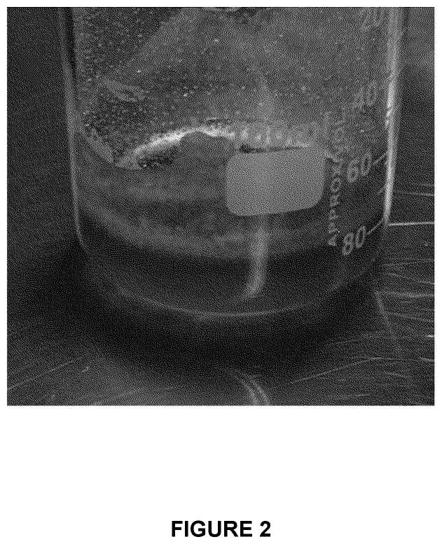

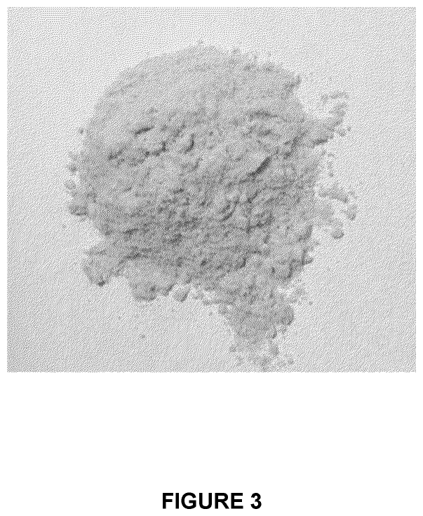

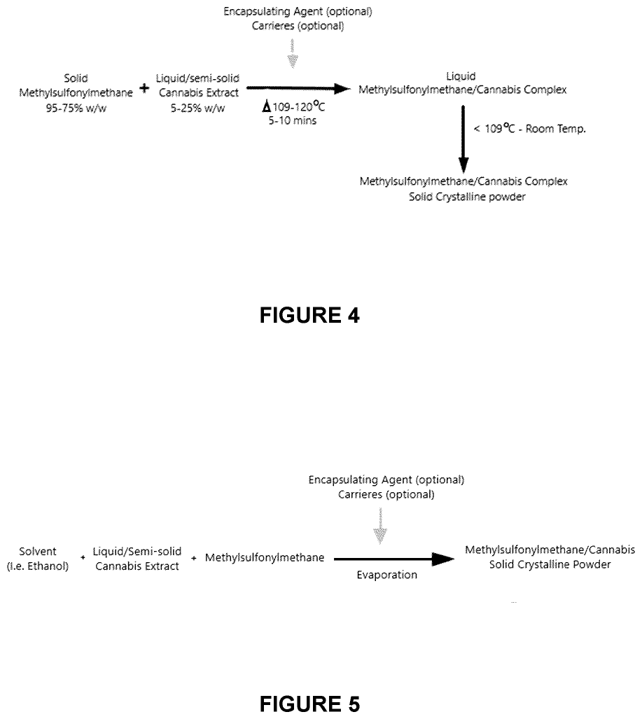

### DETAILED DESCRIPTION OF THE INVENTION

As used herein, the term “about” refers to a +/−10% variation from the nominal value. It is to be understood that such a variation is always included in a given value provided herein, whether or not it is specifically referred to.

The terms “botanicals” and “botanical material” as employed herein refer to materials including plant materials (including but not limited to leaves, stems, buds, bark, roots, and flowers of herbaceous and nonherbaceous plants), algae, macroscopic fungi, and combinations thereof, in accordance with the use of these terms in the document Botanical Drug Development, Guidance for Industry, December 2016, US Department of Health and Human Services, Food and Drug Administration Centre for Drug Evaluation and Research.

The term “botanical extract” refers to extracts of botanical materials obtained by one or more of the following processes: pulverisation, decoction, distillation, expression, aqueous extraction, ethanolic extraction, or other similar processes.

The term “Cannabis plant(s)” encompasses wild type Cannabis sativa and also variants thereof, including those which naturally contain different amounts of the individual cannabinoids, Cannabis sativa subspecies indica including the variants var. indica and var. kafiristanica, Cannabis indica, as well as plants which are the result of genetic crosses, self-crosses or hybrids thereof.

The term “Cannabis plant material” is to be interpreted accordingly as encompassing plant material derived from one or more Cannabis plants, including leaves, buds, flowers, and stems.

In the context of this application, the terms “Cannabis extract” or “extract from a Cannabis plant”, which are used interchangeably, encompass extracts of Cannabis plant material obtained by one or more of the following processes: pulverisation, decoction, expression, maceration, percolation, aqueous extraction, extraction with solvents such as C₁ to C₅ alcohols (e.g., ethanol), Norflurane (HFA134a), HFA227 and liquid carbon dioxide under pressure, or other similar processes. Cannabis extracts can include primary extracts prepared by such processes, which may be further purified for example by supercritical or subcritical extraction, vaporisation and chromatography. When solvents such as those listed above are used, the resultant extract may contain non-specific lipid-soluble material, which can optionally be removed by a variety of processes including “winterisation”, which involves chilling to −20° C., followed by filtration to remove waxy ballast, extraction with liquid carbon dioxide and by distillation.

Preferred “Cannabis extracts” include those which are obtainable by using any of the methods or processes as are known in the art for preparing extracts from Cannabis plant material. The extracts are preferably substantially free of waxes and other non-specific lipid soluble material but preferably contain substantially all of the cannabinoids naturally present in the plant, most preferably in substantially the same ratios in which they occur in the intact Cannabis plant.

Methylsulfonylmethane, or MSM, has been given a “GRAS” designation (Generally Recognized As Safe), and has been approved for use in pharmaceuticals, food, and cosmetics. It is also referred to as dimethyl sulfone or methyl sulfone.

The term “matrix” or “botanical extract/MSM matrix”, as used in the present specification, refers to an evenly distributed solution of a botanical extract in a solid MSM solvent. The term “Cannabis extract/MSM matrix”, as used in the present specification, refers to an evenly distributed solution of a Cannabis extract in a solid MSM solvent.

Unless defined otherwise, all technical and scientific terms used herein have the same meaning as commonly understood by one of ordinary skill in the art to which this invention belongs.

The present invention provides a botanical extract preparation comprising the combination of a botanical extract in a methylsulfonylmethane (MSM) matrix, wherein the preparation is in a powder form that is easy to handle and formulate into convenient and standardized dosage forms. In a preferred embodiment, the botanical extract is a Cannabis extract.

Without intending to be bound by theory, the powdered botanical extract preparation of the present invention can be understood to be an evenly distributed dispersion of the botanical extract in a MSM matrix. In a preferred embodiment, the botanical extract is a Cannabis extract. In such an embodiment, the combination of the Cannabis extract and MSM further provides a composition that has the additional advantage of increased cannabinoid bioavailability upon administration to a subject relative to formulations that do not contain the MSM matrix.

In one embodiment, the composition comprises about 75% to about 95% by weight of MSM and about 5% to about 25% by weight of the botanical extract.

In one embodiment, the powdered botanical extract preparation consists essentially of the botanical extract in a MSM matrix.

In one embodiment, the powdered Cannabis extract preparation consists essentially of the Cannabis extract in a MSM matrix.

In certain embodiments, the powdered botanical extract/MSM matrix may optionally be combined with one or more inactive, neutral compounds/ingredients which can be pharmaceutically acceptable excipients or carriers, including, but not limited to, binders, antioxidants, adjuvants, synergists and/or preservatives.

In certain embodiments, the botanical extract preparations can further include encapsulating agents known in the art (i.e., phospholipids, cyclodextrins, etc.) to encapsulate the actives. These encapsulating agents can be employed with or without the use of aqueous or organic solvents.

In certain embodiments, the powdered botanical extract/MSM preparations may optionally be combined with one or more additional pharmaceutically active components.

The processes of the present invention are particularly suitable for preparing a powdered Cannabis extract preparation that can be handled and manipulated more easily than a Cannabis extract that has not been processed in accordance with the present invention, which is typically in the form of an oily, tar-like substance that is difficult to manipulate, transfer and measure. The present invention therefore also provides formulation processes that offer convenient routes to the Cannabis extract preparations of the present invention.

In accordance with one embodiment of the invention, there is provided a process for preparing the powdered botanical extract preparation that employs the use of molten MSM as a solvent for the botanical extract. In this embodiment, the process involves adding the botanical extract to a molten MSM phase with stirring to provide a botanical extract/MSM mixture, and stirring the resulting mixture until a single phase botanical extract/MSM solution is formed. The botanical extract/MSM single phase solution is allowed to cool until the mixture is solidified and the resulting solid mass is milled to provide the powdered botanical extract preparation, wherein the powdered botanical extract preparation comprises an evenly distributed dispersion of the botanical extract in a MSM matrix. In a preferred embodiment, the botanical extract is a Cannabis extract.

In accordance with another embodiment of the invention, there is provided a process for preparing the powdered botanical extract preparation that employs the use of solvents to initially solubilize the botanical extract. In this embodiment, the process involves pre-dissolving the botanical extract in a suitable solvent to provide a solution of the botanical extract. MSM is added to the botanical extract solution to provide a botanical extract/MSM/solvent mixture, which is stirred until a single phase botanical extract/MSM solution is formed. The solvent is subsequently removed using any suitable solvent removal processes, until a solid botanical extract/MSM residue is formed. The resulting solid botanical extract/MSM residue is milled to provide the powdered botanical extract preparation, wherein the powdered botanical extract preparation comprises an evenly distributed dispersion of the botanical extract in a MSM matrix. In a preferred embodiment, the botanical extract is a Cannabis extract.

In one embodiment, the weight ratio of botanical extract to solvent is from about 1:1 to about 1:4.

In one embodiment, the solvent used to pre-dissolve the botanical extract is a lower alcohol. In a preferred embodiment, the solvent is ethanol.

The processes of the present invention are simple, fast, efficient, and cost-effective, and can be performed using widely available standard commercial equipment. These processes are also easily scalable, and do not require the use of large volumes of organic solvents.

In those embodiments where the powdered botanical extract/MSM preparation is optionally combined with one or more inactive, neutral ingredients or one or more additional pharmaceutically active components, the optional ingredients may be added during the process of preparing the powdered preparation, for example, while the mixture is still in its liquid form and before the solid product has been obtained.

Alternatively, the optional ingredients may be added to the powdered preparation after the process of preparing the powdered preparation has been completed, and before further formulation into suitable dosage forms.

In accordance with the present invention, the powdered botanical extract/MSM preparation can be further incorporated into different dosage forms in accordance with formulation processes as are known in the art.

Some embodiments of the invention are directed to dosage forms that are formulated as solid articles suitable for sublingual or oral administration, such as troches, lozenges, pills, oral dissolving strips, caps or boluses. These solid dosage forms typically comprise additional excipients. They can be prepared by first mixing the powdered botanical extract/MSM preparation of the present invention with one or more suitable excipients followed by molding or compressing the blended mixture. Both hard and chewable lozenges and troches are within the scope of the invention. In one embodiment, the oral dosage form is formulated as capsules in which the powdered botanical extract/MSM preparation is encapsulated in soft or hard gelatin capsules.

The term “carrier” refers to a substance that serves as a vehicle for improving the efficiency of delivery and the effectiveness of a pharmaceutical composition.

The term “excipient” refers to a pharmacologically inactive substance that is formulated in combination with a pharmacologically active ingredient of a pharmaceutical composition and is inclusive of, but not limited to, disintegrants, lubricants, flavorings, bulking agents, binders, fillers, diluents, preservatives, antioxidants, and adjuvants, synergists and products used for facilitating drug absorption or solubility or for other pharmacokinetic considerations.

The term “binder” refers to a substance or compound that promotes, provides or improves cohesion, i.e., a substance that causes the components of a mixture to cohere to form a solid item that possesses integrity.

Some embodiments of the invention are directed to dosage forms that are formulated as topical formulations, including but not limited to creams, ointments and gel, using formulation methods as are known in the art. In one embodiment, the topical formulation is a transdermal patch, using formulation methods and technologies as are known in the art.

Some embodiments of the invention are directed to dosage forms that are formulated as solid articles suitable for administration as vaginal ovules or rectal suppositories, using formulation methods as are known in the art.

Some embodiments of the invention are directed to dosage forms that are formulated as liquids, including but not limited to emulsions, liposomes, dispersions, oils, and tinctures, using formulation methods as are known in the art.

Some embodiments of the invention are directed to dosage forms that are formulated as beverages and edibles, wherein the powdered botanical extract/MSM preparation is incorporated into food and drink products.

Some embodiments of the invention are directed to dosage forms that are formulated as smokeable or vaporizable formulations.

Typical binders that can be used for formulating dosage forms mentioned above include, without limitation, natural, synthetic, or semi-synthetic glycerides, hydrogenated coco-glycerides, mineral oil, cellulose, methyl cellulose and other cellulose derivatives, gelatins, starches, sucrose and sucrose derivatives, lactose and lactose derivatives, silicones (e.g., dimethicone, bis-vinyl dimethicone/dimethicone copolymer), isopropyl myristate, isostearyl palmitate, polyvinylpyrrolidone and derivatives, and polyglycols, e.g., polyethylene glycols (PEGs), polyethylene oxides (POE), methoxpolyethylene glycols, polypropylene glycols, polybutylene glycols or derivatives thereof having a molecular weight that is sufficient to provide the necessary hardness and time for dissolution of the dosage form; for example, the acceptable molecular weight can be within the range of between about 1,000 Daltons and about 8,000 Daltons. In some embodiments, PEG-1450 or PEG-400 can be used. Non-limiting examples of some specific polyglycol derivatives that can be used are: (a) PEG-laureates and dilaureates (e.g., PEG-10-, PEG-12-, PEG-20-, PEG-32-laurates, PEG-20- and PEG-32-dilaurates, PEG-20-glyceryl-, PEG-30-glyceryl- and PEG-40-glyceryl-laurates, PEG-80-sorbitan laurate); (b) PEG-oleates, dioleates and trioleates (e.g., PEG-12-, PEG-15-, PEG-20-, PEG-32, PEG-200- and PEG-400-oleates, PEG-20- and PEG-32-dioleates, PEG-20-trioleate, PEG-25-glyceryl trioleate, PEG-20-glyceryl- and PEG-30-glyceryl-oleates, PEG-40-sorbitan oleate); (c) PEG-stearates and distearates (e.g., PEG-15-, PEG-40-, PEG-100-stearates, PEG-32-distearate and PEG-20-glyceryl stearate) (d) castor, palm kernel, corn and soya oil derivatives of PEG (e.g., PEG-35-, PEG-40- and PEG-60-castor oils, PEG-40-, PEG-50- and PEG-60-hydrogenated castor oils, PEG-40-palm kernel oil, PEG-60-corn oil, PEG-30-soya sterol); (e) other PEG derivatives (e.g., PEG-24- and PEG-30-cholesterol, PEG-25-phytosterol, PEG-6- and PEG-8-caprate/caprylate glycerides, tocopheryl PEG-100 succinate, PEG-15-100 octylphenol products and PEG-10-100 nonylphenol products); (f) other products such as polyglyceryl-10-laurate, POE-9- and POE-23-lauryl ethers, POE-10- and POE-20-oleyl ethers, POE-20-stearyl ether, polysorbate-20 (Tween 20), polysorbate-80 (Tween 80), polyglyceryl-10-oleate, Tween 40, Tween 60, sucrose monostearate, monolaurate and monopalmitate and various products of Poloxamer series.

The dosage forms of the present invention can be formulated, as appropriate, to include disintegrants, including but not limited to starch, cellulose derivatives and alginates, crosslinked sodium carboxymethyl cellulose (corscarmellose sodium), hydroxypropylmethyl cellulose (HPMC), and crosslinked polyvinylpyrrolidone (crospovidone).

The dosage forms of the present invention can be formulated, as appropriate, to include glidants, including but not limited to silicon dioxide, colloidal anhydrous silicon and other silica compounds, and/or and or lubricants including stearic acid and salts thereof, such as magnesium stearate.

Typical excipients that can be used for formulating dosage forms mentioned above include, without limitation, gelatin, sodium saccharin, mannitol, stevioside, peppermint oil, cherry flavor, lemon oil and raspberry flavor.

As stated above, the dosage forms may optionally be formulated to further comprise one or several antioxidants. If antioxidants are used, non-limiting examples of those that can be used include α-tocopherol acetate, acetone sodium bisulfite, acetylcysteine, ascorbic acid, ascorbyl palmitate, butylated hydroxyanisole, butylated hydroxytoluene, cysteine, cysteine hydrochloride, tocopherol natural, tocopherol synthetic, dithiothreitol, monothioglycerol, nordihydroguaiaretic acid, propyl gallate, sodium bisulfite, sodium formaldehyde sulfoxylate, sodium metabisulfite, sodium sulfite, sodium thiosulfate, thiourea and tocopherols.

As stated above, the dosage forms may optionally be formulated to further comprise one or several adjuvants or synergists. If adjuvants or synergists are used, non-limiting examples of those that can be used include citric acid, EDTA (ethylenediaminetetraacetate) and salts, hydroxyquinoline sulfate, phosphoric acid and tartaric acid.

As stated above, the dosage forms may optionally further comprise one or several preservatives. If preservatives are used, non-limiting examples of those that can be used include benzalkonium chloride, benzethonium chloride, benzoic acid and salts, benzyl alcohol, boric acid and salts, cetylpyridinium chloride, cetyltrimethyl ammonium bromide, chlorobutanol, chlorocresol, chorhexidine gluconate or chlorhexidine acetate, cresol, ethanol, imidazolidinyl urea, metacresol, methylparaben, nitromersol, o-phenyl phenol, parabens, phenol, phenylmercuric acetate/nitrate, propylparaben, sodium benzoate, sorbic acids and salts, o-phenylethyl alcohol and thimerosal.

In one embodiment, the amount of inactive ingredients included in a given dosage form can be between about 10% and about 99% by weight of the final dosage form. In one embodiment, the amount of inactive ingredients included in a given dosage form can be between about 20% and about 90% by weight of the final dosage form. In one embodiment, the amount of inactive ingredients included in a given dosage form can be between about 30% and about 70% by weight of the final dosage form. In one embodiment, the amount of inactive ingredients included in a given dosage form can be between about 40% and about 60% by weight of the final dosage form. In one embodiment, the amount of inactive ingredients included in a given dosage form can be between about 75% and about 98% by weight of the final dosage form.

The invention will now be described with reference to specific examples. It will be understood that the following examples are intended to describe embodiments of the invention and are not intended to limit the invention in any way.

### EXAMPLES

### Example 1

### Molten MSM Process

The following is an exemplary process for the formation of a MSM/botanical extract matrix of the present invention, wherein the botanical extract is a Cannabis extract, in accordance with an embodiment of the present invention.

1) In an appropriate vessel equipped with a stirrer set to medium speed, heat 18.5 g MSM at between 109-120° C. until completely melted.

2) Once the MSM has melted, add 1.05 g Cannabis extract.

3) Continue stirring until all of the Cannabis extract has dissolved into the molten MSM.

4) Stir for an additional 5-10 minutes, and then remove from the heat source.

5) Immediately transfer liquid mixture into trays or leave in the vessel to cool to room temperature.

6) Once cooled, liquid mixture solidifies, and the solid product is milled to a powder product having desired particle size.

### Example 2

### Solvent+MSM Process

The following is an exemplary process for the formation of a MSM/botanical extract matrix of the present invention, wherein the botanical extract is a Cannabis extract, in accordance with an embodiment of the present invention.

1) In an appropriate distillation/evaporation vessel (rotary evaporator, vacuum kettle, etc), a solution of 1.05 g of the Cannabis extract dissolved in 4.20 g ethanol (1:4 weight ratio of extract:solvent).

2) 18.5 g MSM is added to the solution produced in step 1.

3) The vessel containing the MSM/Cannabis extract solution is heated, optionally under reduced pressure, to evaporate the solvent to provide a solid residue.

4) The solid residue is collected and milled to a powder product having desired particle size.

### Example 3

### Sublingual Dosage Form

The following is an exemplary sublingual formulation prepared with the ingredients listed in Table 1. The MSM/Cannabis extract matrix was prepared according to the process described in Example 1.

TABLE 1

| Ingredient | % | mg |
|---|---|---|
| MSM/Cannabis Extract Matrix | 37.74 | 100 |
| Silicon Dioxide | 0.38 | 1 |
| Natural Peppermint Flavor | 0.18 | 0.5 |
| Magnesium Stearate | 1.13 | 3 |
| Stearic Acid | 1.02 | 2.7 |
| Sodium Croscarmellose | 0.38 | 1 |
| Mannitol | 59.16 | 156.8 |
| | 100.0 | 265 |

The ingredients are combined and pressed into a sublingual tablet form

### Example 4

### Placebo Dosage Form

A placebo formulation for use as a comparison prepared with the ingredients listed in Table 2.

TABLE 2

| Ingredient | % | mg |
|---|---|---|
| Silicon Dioxide | 0.38 | 1 |
| Natural Peppermint Flavor | 0.18 | 0.5 |
| Magnesium Stearate | 1.13 | 3 |
| Stearic Acid | 1.02 | 2.7 |
| Sodium Croscarmellose | 0.38 | 1 |
| Mannitol | 96.9 | 256.8 |
| | 100.0 | 265 |

The ingredients are combined and pressed into a sublingual tablet form.

### Example 5

### Suppository Dosage Form

The following is an exemplary suppository dosage form prepared with the ingredients listed in Table 3. The MSM/Cannabis extract matrix was prepared according to the process described in Example 1.

TABLE 3

| Ingredient | % | mg |
|---|---|---|
| Semi-synthetic glycerides | 48.39 | 1354.5 |
| Hydrogenated coco-glycerides | 48 | 1315.5 |
| MSM/Cannabis Extract Matrix | 3.61 | 100 |
| | 100 | 2770 |

The ingredients are combined and molded into a rectal suppository dosage form.

### Example 6

### Sublingual Tablet Trial

A sublingual tablet as described in Example 3, formulated to provide an effective THC dosage level of between 2.5-5.0 mg THC (the “test tablet”), was employed in a comparative crossover trial as follows. One test tablet was administered to each of 3 of our laboratory staff members against a placebo arm in a blinded fashion. Each staff member who received a test tablet noticed an onset of various effects within 5-15 minutes of administration, ranging from feeling of wellbeing, euphoria, description of a relaxed/comfortable state, pleasant “goose-bump” sensation of the upper body. The effects were reported to last approximately 1.5 to 2.5 hours in duration.

### Example 7

### Rectal Suppository Trial

A rectal suppository as described in Example 5, formulated to provide an effective THC dosage level of between 2.5-5.0 mg THC (the “test suppository”), was employed in a trial as follows. One test suppository was administered to each of 2 of our laboratory staff members. Both staff members reported onset of effects approximately 30 to 45 minutes after administration. Effects experienced ranged from sense of wellbeing, euphoria, perceived slight increase in heart rate and energy level, and clear-mindedness. Effects lasted beyond 2.5 hours.

It is obvious that the foregoing embodiments of the invention are examples and can be varied in many ways. Such present or future variations are not to be regarded as a departure from the spirit and scope of the invention, and all such modifications as would be obvious to one skilled in the artare intended to be included within the scope of the following claims.

1. A process for preparing a powdered botanical extract preparation, the process comprising the steps of:
a) providing a solid mixture of botanical extract/MSM; and
b) milling the solid botanical extract/MSM mixture to provide the powdered botanical extract preparation,
wherein the powdered botanical extract preparation comprises an evenly distributed dispersion of the botanical extract in a MSM matrix.

2. The process of claim 1, wherein the solid botanical extract/MSM mixture is formed by the following steps:
i) providing a molten methylsulfonylmethane (MSM) phase,
ii) adding a botanical extract to the molten MSM phase to form a liquid mixture of botanical extract/MSM,
iii) stirring the liquid botanical extract/MSM mixture until the botanical extract has dissolved in the MSM to form a single phase botanical extract/MSM solution, and
iv) cooling the single phase botanical extract/MSM solution to form the solid botanical extract/MSM mixture.

3. The process of claim 2, further comprising a step of adding an encapsulating agent or carrier to the botanical extract/MSM mixture prior to cooling.

4. The process of claim 1, wherein the solid botanical extract/MSM mixture is formed by the following steps:
i) providing a solution of a botanical extract in a solvent;
ii) adding methylsulfonylmethane (MSM) to the botanical extract solution to form a botanical extract/MSM/solvent mixture,
iii) stirring the botanical extract/MSM/solvent mixture until a single phase botanical extract/MSM solution is formed,
d) removing the solvent from the homogeneous botanical extract/MSM/solvent phase to form the solid botanical extract/MSM mixture.

5. The process of claim 4, further comprising a step of adding an encapsulating agent or carrier to the botanical extract/MSM mixture prior to removal of the solvent.

6. The process of claim 1, wherein the botanical extract is a Cannabis extract.

7. A powdered botanical extract preparation prepared using the process as defined in claim 1.

8. A powdered Cannabis extract preparation comprising an evenly distributed dispersion of a Cannabis extract in a methylsulfonylmethane matrix.

9. The preparation of claim 8, further comprising one or more pharmaceutically acceptable excipients or carriers.

10. A dosage form comprising a preparation as defined in claim 8, and one or more pharmaceutically acceptable excipients or carriers.

11. The dosage form of claim 10, wherein the dosage form is an oral dosage form selected from a troche, a lozenge, a pill, an oral dissolving strip, a tablet, a capsule, or a bolus.

12. The dosage form of claim 10, wherein the dosage form is an oral dosage form formulated as a beverage or an edible, and wherein the powdered Cannabis extract/MSM composition is incorporated into a foodstuff.

13. The dosage form of claim 10, wherein the dosage form is formulated as a vaginal ovule or a rectal suppository.

14. The dosage form of claim 10, wherein the dosage form is formulated as an emulsion, a liposome, a dispersion, an oil, or a tincture.

15. The dosage form of claim 10, wherein the dosage form is a smokeable or vaporizable formulation.

16. A topical formulation comprising a preparation as defined in claim 8, and one or more pharmaceutically acceptable excipients or carriers.

17. The topical formulation of claim 16, wherein the topical formulation is a cream, an ointment or a gel.

18. The topical formulation of claim 16, wherein the topical formulation is a transdermal patch.

19. The process of claim 2, wherein the botanical extract is a Cannabis extract.

20. The process of claim 5, wherein the botanical extract is a Cannabis extract.
